(19) United States
(12) Patent Application Publication
(10) Pub. No.: US 2009/0010992 A1
(43) Pub. Date: Jan. 8, 2009

(54) Drug formulations for oral transmucosal delivery to pediatric patients

(75) Inventors: Pamela Palmer, San Francisco, CA (US); Thomas Schreck, Portola Valley, CA (US); Stelios Tzannis, Newark, CA (US); Larry Hamel, Mountian View, CA (US); Andrew I. Poutiatine, San Anselmo, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: AceIRx Pharmaceuticals, Inc., Redwood City, CA (US)

(21) Appl. No.: 11/974,007

(22) Filed: Oct. 11, 2007

Related U.S. Application Data
(63) Continuation of application No. 11/825,154, filed on Jul. 3, 2007, now abandoned.

(51) Int. Cl. A61K 9/00; A61K 31/135; A61K 31/33; A61P 1/08; A61P 25/00; A61P 11/06; A61K 31/445; A61K 31/55

(52) U.S. Cl. 424/439; 514/183; 514/220; 514/326; 514/653

(57) ABSTRACT

Improved compositions, methods and systems for oral transmucosal administration of small volume bioadhesive drug dosage forms to pediatric subjects are provided. The drug dosage form is easily administered and may be delivered using a single dose applicator or a device.

### FIELD OF THE INVENTION

The present invention relates to compositions, methods, and systems for delivery of medications to pediatric subjects via the oral transmucosal route.

### BACKGROUND OF THE TECHNOLOGY

Oral administration is the most prevalent method of delivering medications to pediatric patients. The medication is generally incorporated into a tablet, capsule, or a liquid and swallowed. Oral administration is generally painless and simple for most patients.

However, oral administration suffers from several disadvantages in particular for the pediatric population. Pediatric patients frequently have difficulty swallowing pills and other solid dosage forms, and may refuse to swallow a liquid medication. As a result, the pediatric population poses unique challenges for non-invasive drug delivery.

In order to avoid some of the disadvantages of oral administration, injection is frequently used, typically intramuscular (IM) or intravenous (IV). Injecting a drug results in rapid entry of the drug into the patient's bloodstream and less drug is usually necessary to achieve a rapid therapeutic benefit as compared to orally administered drugs.

If a child is hospitalized and an IV catheter is in place, medications can easily be administered IV. However, sedating a child in order to place an IV catheter, or administering medications to a small child when IV access is not available can be a challenge with currently available medications.

To overcome these challenges in the past, rectal suppositories or oral liquids have been utilized. Rectal suppositories can result in erratic uptake of medications and are unpleasant to administer.

In addition, the efficiency of absorption of a drug following oral administration can be low because of metabolism within the GI tract and first-pass metabolism within the liver resulting in relatively lengthy onset times or erratic absorption characteristics that are not well suited to control acute disorders. The majority of oral dosage forms on the market are designed for GI delivery.

Further, there is a substantial delay between the time of oral administration and the time that the therapeutic effect of the drug begins. The oral route of administration can thus result in erratic plasma levels of drugs as well as a delayed onset of action.

Anxiety and stress often increase this delay, in particular for pediatric patients. This is particularly problematic when treatment of acute conditions, such as pain, anxiety, nausea/vomiting, etc. is both necessary and urgent. For many applications where immediate relief from pain or treatment of a serious medical condition where immediate effectiveness of the drug is required, this delay is unacceptable.

Oral transmucosal delivery offers a number of advantages in that it can provide a shorter onset time and more consistent time (Tₘₐₓ) to maximal plasma concentration (Cₘₐₓ) than oral delivery, in particular for lipophilic drugs. This is because the drug rapidly passes directly and efficiently through the relatively permeable epithelium of the highly vascularized mucosal tissue to the plasma, thus rapidly reaching the circulation while avoiding the slower, often inefficient and variable GI uptake. In addition, due to the avoidance of the first-pass metabolism and avoidance of inefficient drug absorption though the gut, oral transmucosal drug uptake also improves bioavailability. It is therefore advantageous for a drug to be delivered through the mucus membranes of the oral cavity, (e.g., via the sublingual route), when rapid onset, consistent Tₘₐₓ and Cₘₐₓ are desired.

Various solid dosage forms, such as sublingual tablets, troches, lozenges, lozenges-on-a-stick, chewing gums, and buccal patches, have been used to deliver drugs via the oral mucosal tissue.

Although a number of oral mucosal drug delivery systems have been described, there remains a need for improved formulations for use in drug delivery to children that allows for a relatively undetectable dosage form that is easily administered to and accepted by pediatric patients. The present invention addresses this need.

### BRIEF SUMMARY OF THE INVENTION

The invention provides small volume bioadhesive drug dosage form compositions for oral transmucosal administration to a pediatric subject, and methods of using such compositions to treat a symptomatic medical condition in a pediatric subject.

The drug dosage form has a volume of less than 30 microliters or a mass of less than 30 mg and comprises a drug formulation containing a predetermined amount of a pharmaceutically active drug and a bioadhesive material, where the bioadhesive material provides for adherence to the oral mucosa of the subject.

In some embodiments, the oral transmucosal administration is sublingual or buccal administration.

The invention provides a drug dosage form containing a pharmaceutically active drug effective to treat an acute medical condition selected from the group consisting of anxiety (e.g., pre-procedural anxiety), pain (e.g., acute or post-operative pain), nausea, vomiting or asthma.

A drug dosage form of the invention may also be used for sedation (e.g., pre-procedural sedation) of a pediatric subject.

A drug dosage form of the invention for treatment of pre-procedural anxiety comprises the combination of an anxiolytic or sedative agent and an agent for treatment of pain in a single dosage form. Such a dosage form includes at least one benzodiazepine or non-benzodiazepine anxiolytic or sedative agent in combination with at least one opioid or non-opioid analgesic. 100191 The benzodiazepine agent in a dosage form of the invention is selected from the group consisting of triazolam, alprazolam, midazolam, temazepam, estazolam, flurazepam, nitrazepam, bromazepam, halazepam and clonazepam or similar benzodiazepine.

The non-benzodiazepine agent in a dosage form of the invention is selected from the group consisting of zolpidem tartrate, zaleplon, and eszopiclone.

The opioid agent in a dosage form of the invention is selected from fentanyl, sufentanil, alfentanil, lofentanil, carfentanil, remifentanil, trefentanil, and mirfentanil.

A drug dosage form of the invention for treatment of pre-procedural anxiety may comprise: (1) about 50 mcg to about 1000 mcg of triazolam and from about 1 mcg to about 50 mcg of sufentanil per dosage form; (2) about 10 mcg to about 1000 mcg of triazolam and from about 10 mcg to about 500 mcg of fentanyl per dosage form; (3) about 1 mcg to about 50 mcg of sufentanil per dosage form; or (4) about 0.5 mg to about 10 mg of midazolam and from about 10 mcg to about 500 mcg of fentanyl per dosage form.

The invention further provides a drug dosage form for treatment of pain where the pharmaceutically active drug is selected from fentanyl, sufentanil, alfentanil, lofentanil, carfentanil, remifentanil, trefentanil, and mirfentanil.

A drug dosage form of the invention for treatment of pain may comprise: (1) from about 1 mcg to about 50 mcg of sufentanil; or (2) from about 10 mcg to about 500 mcg of fentanyl.

The invention further provides a drug dosage form for treatment of nausea or vomiting where the pharmaceutically active drug is selected from ondansetron, dolasetron and granisetron, droperidol, prochlorperazine, metaclopramide and cannabinoid receptor agonists.

A drug dosage form of the invention for treatment of nausea or vomiting may comprise from about 500 mcg to about 20 mg of ondansetron.

The invention further provides a drug dosage form for treatment of asthma where the pharmaceutically active drug is selected from short-acting beta2-adrenergic receptor agonists, such as salbutamol, terbutaline, albuterol, fenoterol and orciprenaline.

A drug dosage form of the invention for treatment of nausea or vomiting may comprise from about 10 mcg to about 2 mg of terbutaline.

The invention also provides disposable single dose applicators (SDAs) for dispensing a small volume bioadhesive drug dosage form as further described below.

In one aspect of the invention, the subject is an infant or child for whom administering the medication would otherwise be difficult or uncomfortable.

Another aspect of the invention provides methods for treating a pediatric subject exhibiting a symptomatic medical condition, by administering a drug dosage form via the oral transmucosal route such that the treatment is easily administered since it is not easily detected by the subject, therefore providing effective drug administration to treat symptomatic medical conditions.

### BRIEF DESCRIPTION OF THE FIGURES

FIG. 1 is a graphic depiction of sufentanil plasma concentrations following sublingual administration of an exemplary sufentanil NanoTab™ formulation compared to intravenous sufentanil administration (n=3) in a healthy, conscious Beagle dog model, as detailed in U.S. Ser. No. 11/650,174, expressly incorporated by reference herein. Error bars represents standard errors around the mean (SEM).

FIG. 2 is a graphic depiction of sufentanil plasma concentrations following intravenous dosing or sublingual single dose administration of three different strengths of sufentanil NanoTabs® in healthy human volunteers (n=12).

FIGS. 3A and 3B are schematic depictions of exemplary single dose applicators.

FIGS. 4A-C provide an illustration of one type of single dose applicator and its use in delivering a dosage form to a subject.

FIGS. 5A-F provide an illustration of six additional single dose applicators.

FIG. 6 provides an illustration of an additional type of single dose applicator, wherein the drug dosage form is a film or strip and multiple single dose applicators showing a plurality of single dose applicators housed in a dispenser.

FIGS. 7A-B provides illustrations of two stages of use of one embodiment of a single dose applicator.

### DETAILED DESCRIPTION

The present invention is directed to compositions, methods and systems for the treatment of symptomatic medical conditions in pediatric subjects by delivery of a drug via the oral mucosa using a small volume dosage form.

The invention finds utility: (1) in the inpatient setting in the context of hospital-based procedures, (2) in doctor's offices, (3) in clinics, (4) in the outpatient setting for use in place of oral or IV drugs for treatment of conditions such as pain, anxiety (e.g., pre-procedural anxiety), sedation (e.g., pre-procedural sedation), nausea and/or vomiting and asthma and (5) in the “field” for treatment of any acute medical condition, such as pain and/or anxiety.

The following disclosure describes the compositions, methods and systems which constitute the invention. The invention is not limited to the specific drug dosage forms, methodology or medical conditions described herein, as such may, of course, vary. It is also to be understood that the terminology used herein is for the purpose of describing particular embodiments only, and is not intended to limit the scope of the present invention.

It must be noted that as used herein and in the appended claims, the singular forms “a” “and”, and “the” include plural references unless the context clearly dictates otherwise. Thus, for example, reference to “a drug formulation” includes a plurality of such formulations and reference to “a drug delivery device” includes systems comprising drug formulations and devices for containment, storage and delivery of such formulations.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference in their entirety for the purpose of describing and disclosing the compositions and methodologies which are described in the publications which might be used in connection with the presently described invention. The publications discussed herein are provided solely for their disclosure prior to the invention filing date of the present application. Nothing herein is to be construed as an admission that the invention is not entitled to antedate such a disclosure by virtue of prior invention.

### DEFINITIONS

The term “anxiolytic” as used herein refers to a drug prescribed for the treatment of symptoms of anxiety.

The term “formulation” or “drug formulation” or “dosage form” as used herein refers to a composition containing at least one therapeutic agent or medication for delivery to a subject. The dosage form comprises a given “formulation” or “drug formulation” and may be administered to a patient in the form of a lozenge, pill, tablet, capsule, membrane, strip, paper, liquid, patch, film, gel, spray or other form.

The terms “drug”, “medication”, “pharmacologically active agent” and the like are used interchangeably herein and generally refer to any substance that alters the physiology of an animal. A dosage from comprising a formulation of the invention may be used to deliver any drug that may be administered by the oral transmucosal route. The term “drug” as used herein with reference to a formulation of the invention means any “drug”, “active agent”, “active”, “medication” or “therapeutically active agent” that can be effectively administered by the oral transmucosal route.

The term “drug” may be used interchangeably herein with the term “therapeutic agent” or “medication”. It will be understood that a “drug” formulation of the invention may include more than one therapeutic agent.

The term “active agent” or “active” may be used interchangeably herein with the term “drug” and is used herein to refer to any therapeutically active agent.

The term “congener” as used herein refers to one of many variants or configurations of a common chemical structure.

The term “subject” includes any pediatric subject, in which treatment, such as management of pain, anxiety (e.g., pre-procedural anxiety), nausea and/or vomiting, or sedation (e.g., pre-procedural sedation) is desired.

The term “pediatric subject” as used herein refers to a subject who is an infant or a child. The term “pediatrics” as used herein is the branch of medicine that deals with the care of infants and children and the treatment of their diseases.

The term “symptomatic medical condition” is used herein with reference to pain, anxiety (i.e. pre-procedural anxiety), nausea and asthma.

The term “mucosal membrane” refers generally to any of the mucus-coated biological membranes in the body. Absorption through the mucosal membranes of the oral cavity is of particular interest. Thus, buccal, sublingual, gingival and palatal absorption are specifically contemplated by the present invention.

The term “transmucosal” delivery of a drug and the like is meant to encompass all forms of delivery across or through a mucosal membrane. In particular, “oral transmucosal” delivery of a drug includes delivery across any tissue of the mouth, pharynx, larynx, trachea, or upper gastrointestinal tract, particularly including the sublingual, gingival and palatal mucosal tissues.

The terms “oral dosage form” and “oral transmucosal dosage form” may be used interchangeably herein and refer to a dosage form for use in practicing the present invention, which comprises a drug formulation as described herein. The oral dosage form is typically a “sublingual dosage form”, but in some cases other oral transmucosal routes may be employed. The invention relies upon such oral dosage forms to provide controlled delivery of drugs across the oral mucosa by controlling the formulation design immediate, intermediate and sustained release of drugs can be achieved, as described below. The dosage form is a substantially homogeneous composition which comprises active ingredients and one or more of mucoadhesives (also referred to herein as “bioadhesives”) that provide for adherence to the mucosa of the mouth of a patient, binders for binding the excipients in a single dosage form, one or more hydrogel-forming excipients, one or more bulking agents, one or more lubricants, as well as other excipients and factors that affect dissolution time or drug stability. The drug formulations of the invention are neither effervescent nor do they comprise an essentially water-free, ordered mixture of microparticles of drug adhered to the surface of carrier particles, where the carrier particles are substantially larger than the microparticles of drug. In one aspect, the present invention provides small-volume oral transmucosal drug delivery dosage forms.

The term “NanoTab™” as used herein refers to a small volume dosage form that has a volume of from about 0 ul (microliters) to about 100 ul and a mass of from about 0 mg (milligrams) to about 100 mg. A NanoTab™ of the invention may or may not have bioadhesive characteristics and is a drug-containing dosage form which may have characteristics of hydrogel-forming or eroding tablets.

The term “oral transmucosal drug delivery” as used herein refers to a dosage form wherein drug delivery occurs substantially via the transmucosal route and not via swallowing followed by GI absorption. The formulations of the current invention are designed to provide for a drug dissolution rate that allows for maximal delivery via the oral mucosa, and also provide controlled delivery rates across the oral mucosa typically via placement of the dosage form within the sublingual space.

As used herein, “sublingual”, means literally “under the tongue” and refers to a method of administering substances via the mouth in such a way that the substances are rapidly absorbed via the blood vessels under the tongue rather than via the digestive tract. Among the various transmucosal sites, the mucosa of the sublingual v is found to be the most convenient and easily accessible site for the delivery of therapeutic agents for both local and systemic delivery as controlled release dosage forms because it of its abundant vascularization and the near absence of Langerhans cells. Direct access to the systemic circulation through the internal jugular vein bypasses the hepatic first pass metabolism leading to high bioavailability. Further, owing to the highly vascularized nature of the sublingual mucosal membrane and the reduced number of epithelial cell layers compared to other mucosal membranes, absorption of therapeutic substances occurs rapidly, thus allowing for direct access to the systemic circulation and thus enable quick onset of action while avoiding complications of oral administration.

The term “disintegration” may be used interchangeably herein with the term “erosion” and means the physical process by which a dosage form breaks down and pertains to the physical integrity of the dosage form alone. This can occur in a number of different ways including breaking into smaller pieces and ultimately, fine and large particulates or, alternatively, eroding from the outside in until the dosage form has disappeared.

The term “dissolution” as used herein means the process by which the active ingredient is dissolved from the dosage form in the presence of a solvent, in vitro, or physiological fluids in vivo, e.g., saliva, irrespective of the mechanism of release, diffusion, erosion or combined erosion and diffusion.

The term “bioadhesion” as used herein refers to the process of adhesion of a dosage forms to a biological surface or mucosal membrane, for example the oral mucosa.

The expression “mucoadhesion” is used herein to refer to adhesion to mucosal membranes which are covered by mucus, such as those in the oral cavity and is used interchangeably herein with the term “bioadhesion” which refers to adhesion to any biological surface.

The term “therapeutically effective amount” means an amount of a therapeutic agent, or a rate of delivery of a therapeutic agent (i.e., amount over time), effective to facilitate a desired therapeutic effect, such as pain relief. The precise desired therapeutic effect (e.g., the degree of pain relief, and source of the pain relieved, etc.) will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

The term “Tₘₐₓ” as used herein means the time point of maximum observed plasma concentration.

The term “Cₘₐₓ” as used herein means the maximum observed plasma concentration.

The term “AUC” as used herein means “area under the curve” in a plot of concentration of drug in plasma versus time. AUC is often given for the time interval zero to infinity, however, clearly plasma drug concentrations cannot be measured ‘to infinity’ for a patient so mathematical approaches are used to estimate the AUC from a limited number of concentration measurements. In a practical sense, the AUC (from zero to infinity) represents the total amount of drug absorbed by the body, irrespective of the rate of absorption. This is useful when trying to determine whether two formulations of the same dose release the same dose of drug to the body. The AUC of a transmucosal dosage form compared to that of the same dosage administered intravenously serves as the basis for a measurement of bioavailability.

The term “bioavailability” as used herein means “percent bioavailability” and represents the fraction of drug absorbed from the test article as compared to the same drug when administered intravenously. It is calculated from the AUC_{∞} of the test article following delivery from the intended route versus the AUC_{∞} for the same drug after intravenous administration. It is calculated from the equation: Bioavailability (%)=AUC_{∞}(test article)/AUC_{∞}(intravenous route/article). This is an important term that establishes the relative fraction of the drug absorbed via the test route (or article) versus the maximum possible amount absorbed via the intravenous route.

As used herein, when a drug formulation is said to “adhere” to a surface, such as a mucosal membrane, it is meant that the formulation is in contact with said surface and is retained on the surface without the application of an external force. Adhesion is not meant to imply any particular degree of sticking or bonding, nor is it meant to imply any degree of permanency.

I. Pediatric Subjects

The dosage forms and methods of the invention find particular utility in pediatric applications, since the comfortable and secure nature of the dosage form will allow pediatric patients to readily accept this mode of therapy and will reliably deliver the drug transmucosally. Specific examples include situations when: (1) IV access is not available; (2) initiating IV access would require sedation; (3) a pediatric patient can not or will not swallow a pill; (4) a pediatric patient is NPO (no oral intake allowed, food, liquids or medications); (4) pre-procedural sedation or a relatively rapid onset of the medication is required and cannot be achieved by oral administration; and (5) the pediatric patient is not able to use an inhaled route or other non-invasive route of administration effectively.

Specific examples of situations where oral transmucosal drug delivery to pediatric patients provides advantages over current drug administration methods include, but are not limited to, any treatment of an acute condition for which IV administration is not readily available or is inconvenient and oral delivery results in delayed onset or is ineffective, such as pain, anxiety (e.g., pre-procedural anxiety), sedation (e.g., pre-procedural sedation), nausea and/or vomiting and asthma. Oral transmucosal drug delivery for treatment of pre-procedural anxiety is described in U.S. Ser. No. 60/922,084, expressly incorporated by reference herein.

The oral transmucosal route offers many theoretical advantages for drug administration. Absorption occurs directly into the systemic circulation thereby avoiding first-pass metabolism and the effect of gastrointestinal and liver enzymes which can metabolize the active drug. This route of administration provides both consistently higher and less erratic bioavailability than the oral-gastrointestinal (GI) route and is also is advantageous since a transition from inpatient to outpatient use of the medication can occur with no change in the route of administration. This is not true for intravenous administration.

Placement of a dosage form in the sublingual space or the buccal space between the cheek and gums requires cooperation on the part of the pediatric patient. Even when sublingual liquids are administered in children, they often either spit out the drug or swallow it, thereby essentially losing any transmucosal uptake. The small volume oral dosage forms of the invention are easily administered to a pediatric patient and are unlikely to be detected by the child after placement in a sublingual or buccal location due to the small size of the dosage form. Hence, the present invention provides a means for effective administration of a drug to a pediatric patient that has a greater probability of both acceptance by the patient and a more rapid and consistent onset of efficacy than currently available drug dosage forms for administration to pediatric patients.

II. Treatment of Pain

Pain control is attempted in many clinical and non-clinical settings using a number of interventions, which generally include: IV administration, e.g., using an IV patient-controlled analgesia (PCA) pump, continuous epidural infusion, other types of acute pain control, palliative care pain control, and home health patient pain control. These methods meet with varying degrees of success with respect to duration of control, ease of treatment and safety versus side effects.

Opioids are powerful analgesics and are utilized to treat both acute and chronic pain of moderate to severe intensity, however, they can also have severe respiratory depressive effects if not used appropriately and they also are known to produce pruritis, nausea and vomiting during acute use as well as physical dependence, and tolerance with long-term use.

Opioids are widely used for the treatment of pain, and are generally delivered IV, orally, epidurally, transdermally, rectally and intramuscularly. Whereas there are many opioid products available for treatment of chronic pain, there are relatively few options for treatment of acute, fast-onset, intense pain. Morphine and its analogues are commonly delivered IV and are effective against severe, chronic and acute pain.

In the hospital setting, most patients are given IV opioids to provide rapid relief from pain. Transmucosal administration of opioids has been used to treat pre-procedural anxiety, especially in children, however, the increased dose required for sedation is higher than required for analgesic purposes and may result in an increased incidence of respiratory depression and nausea and vomiting, which can delay discharge from the post-surgical recovery room (Clin. Pharmacol and Therapeutics 59:341, 1996).

On the other hand, inadequate sedation and pain control has been shown to have negative implications for pediatric patients. Inadequate pain control during oncology procedures leads to significantly increased pain scores for subsequent painful procedures. Post-traumatic stress disorder can occur after procedures or stressful medical experiences that are not accompanied by appropriate pain control or sedation (Zempsky et al., Pediatrics Vol. 114 No. 5 November 2004, pp. 1348-1356).

### Fentanyl Congeners

Fentanyl (N-(1-phenethyl-4-piperidyl)-N-phenyl-propanamide) has an analgesic potency of about 80 times that of morphine. Fentanyl and its congeners are mu opioid agonists that were originally developed as anesthesia agents, and are often administered intravenously due to rapid onset of analgesia. Following IV administration, the analgesic action of fentanyl is more prompt and less prolonged than that of morphine and meperidine.

Sufentanil (N-[(4-(Methoxymethyl-1-(2-(2-thienyl)ethyl)-4-piperidinyl)]-N-phenylpropanamide), is used as a primary anesthetic, to produce balanced general anesthesia in cardiac surgery, for epidural administration during labor and delivery and has been administered experimentally in both intranasal and liquid oral formulations. A commercial form of sufentanil used for IV delivery is the SUFENTA FORTE® formulation. This liquid formulation contains 0.075 mg/ml sufentanil citrate (equivalent to 0.05 mg of sufentanil base) and 9.0 mg/ml sodium chloride in water. It has a plasma elimination half-life of 148 minutes, and 80% of the administered dose is excreted in 24 hours.

In one aspect of the invention, a small volume bioadhesive drug dosage form comprising an opioid is provided for oral transmucosal administration to a pediatric subject.

The drug delivery dosage forms or formulations of the invention contain from about 0.25 to about 100 mcg of sufentanil per dosage form for oral transmucosal delivery. In one exemplary embodiment of the invention, each dosage form contains from about 0.25 to about 50 mcg of sufentanil, in combination with one or more other therapeutic agents or drugs.

Exemplary formulations of the invention for administration to pediatric patients contain from about 0.25 to about 100 mcg of sufentanil per dosage form. For example, a formulation of the invention for administration to children may contain about 0.25, 0.5, 1, 2.5, 4, 5, 6, 8, 10, 15, 20, 40, 60 or 100 mcg of sufentanil for oral transmucosal delivery. It follows that for pediatric patients, an exemplary dose range is from at least about 0.02 mcg/kg to about 2 mcg/kg with a preferable range of from about 0.05 to about 1 mcg/kg. Higher doses are restricted to opioid-tolerant children.

Exemplary dosage forms of the invention for administration to pediatric patients contain from about 10 to about 500 mcg of fentanyl per dosage form. For example, a dosage form of the invention for administration to children may contain about 10, 20, 30, 40, 50, 60, 80, 100, 150, 300, 450, or 500 of fentanyl for oral transmucosal delivery, the higher doses restricted to opioid-tolerant children. Fentanyl may be provided in combination with one or more other therapeutic agents or drugs.

Congeners of sufentanil and fentanyl also find use in the compositions and methods of the invention, examples of which include remifentanil, alfentanil, lofentanil, carfentanil, trefentanil, and mirfentanil.

Remifentanil is a potent fentanyl congener that is metabolized much more rapidly than fentanyl or sufentanil, but may be suitable for treatment of acute pain when delivered via a sustained-release formulation. A dosage form for use in practicing the invention, e.g., a NanoTab™ typically comprises from about 0.25 mcg to 99.9 mg of remifentanil, which may be provided in combination with one or more other therapeutic agents or drugs. The dose ranges for the remifentanil formulation may include 0.1 mcg/kg-50 mcg/kg over a time period of 20 minutes, for example, for both adult and pediatric patients. These dosages may be repeated at appropriate time intervals, which may be shorter than the time intervals for fentanyl or sufentanil.

Alfentanil is also a potent fentanyl congener that is rapidly metabolized but may be suitable for use in a sustained-release formulation. A dosage form for use in practicing the invention typically comprises from about 10 mcg to about 10 mg of alfentanil, which may be provided in combination with one or more other therapeutic agents or drugs. Appropriate dosing of alfentanil may be in the range of 1 mcg/kg-2000 mcg/kg over 20 minutes, for example, for both adult and pediatric patients. These dosages may be repeated at appropriate time intervals, which may be shorter than the time intervals for fentanyl or sufentanil.

The dosage forms of the invention contain from about 10 mcg to about 10 mg of alfentanil per dosage form for oral transmucosal delivery. As will be understood by those of skill in the art, the dose will be on the low end of the range for children and the high end of the range for adults dependent upon body mass, in particular when administered long term to opioid-tolerant adults.

Exemplary dosage forms of the invention for administration to children (pediatric patients) contain from about 10 to about 6300 mcg of alfentanil per dosage form which may be provided in combination with one or more other therapeutic agents or drugs. For example, a dosage form of the invention for administration to children may contain about 10, 25, 50, 130, 210, 280, 310, 420, 600, 780, 1050, 2100, 3000 or 6300 mcg of alfentanil for oral transmucosal delivery.

Exemplary dosage forms of the invention for administration to adults contain from about 70 to about 10000 mcg of alfentanil per dosage form which may be provided in combination with one or more other therapeutic agents or drugs. For example, a dosage form of the invention for administration to adults may contain about 70, 140, 160, 210, 280, 310, 420, 600, 780, 1050, 2100, 3000, 6300 or 10000 mcg or more of alfentanil for oral transmucosal delivery.

Lofentanil, carfentanil, trefentanil, and mirfentanil are also potent fentanyl congeners that are rapidly metabolized and may be suitable for use in a sustained-release formulation.

More specifically, the invention provides a dosage form which comprises an opioid drug in an amount selected from the group consisting of from about 0.25 μg to 99.9 mg of lofentanil, from about 0.25 μg to 99.9 mg of carfentanil, from about 0.25 μg to 99.9 mg of carfentanil, from about 0.25 μg to 99.9 mg of remifentanil, from about 0.25 μg to 99.9 mg of trefentanil, from about 0.25 μg to 99.9 mg of mirfentanil, respectively, which may be provided in combination with one or more other therapeutic agents or drugs.

As will be understood by those of skill in the art, the dose will be on the low end of the range for children and the high end of the range for adults dependent upon body mass, in particular when administered long term to opioid-tolerant adults.

Following delivery of a single dose of a fentanyl-, sufentanil-, remifentanil-, alfentanil-, lofentanil-, carfentanil-, trefentanil-, or mirfentanil containing dosage form of the invention to a human subject, the plasma level of fentanyl, sufentanil, remifentanil, alfentanil, lofentanil, carfentanil, trefentanil, or mirfentanil may reach a maximum level between 0 and 60 minutes, between 5 and 50 minutes or between 10 and 40 minutes after administration.

III. Anxiety

Anxiety for pediatric subjects often is triggered by a specific condition or situation, such as an intense fear in an emergency medical situation, fear prior to a medical or dental procedure, fear of flying in an airplane, fear of enclosed spaces or being trapped, fear of heights, etc. This is particularly problematic in the pediatric situation as infants and children often do not know that their fear of the object or situation is excessive or unreasonable.

Anti-Anxiety Medications

A number of classes of drugs are used to treat anxiety disorders, including but not limited to, benzodiazepines, beta adrenergic receptor blockers, miscellaneous anxiolytics, monoamine oxidase inhibitors, selective serotonin reuptake inhibitors (SSRIs), serotonin-norepinephrine reuptake inhibitors (SNRIs) and other anxiolytics. Certain drug classes have greater effectiveness for specific anxiety disorders than others. For an acute anxiety attack, short-term treatment with benzodiazepines is a standard treatment. More chronic episodes of anxiety are typically treated by administration of SSRIs, SNRIs or buspirone. In other situations, tricyclic antidepressants, beta-blockers, and, rarely, monoamine oxidase inhibitors are prescribed alone or in combination with other drugs to control anxiety.

Benzodiazepines

Exemplary drugs of the benzodiazepine class include but are not limited to triazolam, midazolam, temazepam, estazolam, alprazolam, diazepam and lorazepam, and are usually taken orally.

Oral benzodiazepines act fairly rapidly (within 1-2 hours), with a limited number of side effects which can include agitation, worsened anxiety, confusion, impaired memory, lack of coordination, speech difficulties, and others.

Using the sublingual route to deliver benzodiazepines pre-procedurally has resulted in effective sedation as demonstrated by a number of published studies. See, e.g., Stopperich P S, et al., Anesth Prog. 1993;40(4):117-21; Tweedy et al., J Clin Psychopharmacol. 2001, 21(3):268-72; Scavone J M et al., J Clin Pharmacol. March 1986, 26(3):208-10; Kontinen et al., Canadian Journal of Anesthesia, Vol 40, 829-834, 1993; and McCann and Kain, Anesthesia & Analgesia, 93:98-105, 2001.

Non-Benzodiazepine Sedatives

Exemplary sedative drugs of the non-benzodiazepine class include but are not limited to ketamine, Ambien (zolpiderm, also known as zolpiderm tartrate) in the imidazopyridine class, SONATA® (zaleplon) in the pyrazolopyrimidine class and LUNESTA (eszopiclone) the cyclopyrrolone class.

Physicians and nurses are often required to perform painful or frightening procedures on children. Children may view needle sticks as the worst source of pain and fear in the hospital setting. In an effort to minimize the pain of needle sticks, the use of a mixture of lidocaine and prilocaine (EMLA) has become standard practice in many children's hospitals. Unfortunately, EMLA requires at least 60 minutes to be fully effective and reportedly may cause vasoconstriction, leading to difficult vein cannulation.

Pre-procedural anxiety and successful sedation have been inversely correlated. It has been shown that children with low anxiety are 3.8 times more likely to be successfully sedated (Schreiber K M et al., Am J Emerg Med. July 2006;24(4):397-401). In addition, high levels of anxiety may result in more difficult and painful procedures.

When IV access is not available, often either a benzodiazepine, such as oral or intranasal midazolam, or an opioid, such as intranasal sufentanil, is used for pre-procedural sedation (Karl et al., Anesthesiology, 76:209-15, 1992). There are disadvantages of using only a single agent for pre-procedural sedation. Benzodiazepines, especially when given via the oral route, can have a delayed and erratic onset which results in delayed post-procedural recovery (Viitanen et al., Anesthesia & Analgesia, 89:75-9, 1999; Viitanen et al, Canadian Journal of Anaesthesia, 46:766-71, 1999). Also, there is no direct analgesic effect of benzodiazepines or most sedatives, which can result in increased anxiety and agitation due to under-treated pain. On the other hand, using opioids alone to provide pre-procedural sedation can result in episodes of respiratory depression and post-procedural nausea and vomiting (Friesen and Lockhart, Anesthesiology, 76:46-51, 1992; Karl et al., Anesthesiology, 76:209-15, 1992). Further, when the transmucosal formulation of fentanyl, the Fentanyl Oralet (fentanyl 10-15 micrograms/kg), was administered to 3-10 year old patients, followed by a determination of bioavailability, estimated fentanyl bioavailability was 36.1±0.4%, suggesting that many children swallowed a large fraction of the dose (Wheeler M, et al., Paediatr Anaesth. September 2002; 12(7):594-9). The fentanyl bioavailability observed in the Wheeler study was less than that observed in adults who received a transmucosal fentanyl lozenge (Actiq) (50±11%) and the same as that observed for adults who were administered oral fentanyl solution (32±10%) (Wheeler M, et al., 2002).

Therefore, there are significant advantages for pre-procedural sedation in combining both a sedative agent, such as a benzodiazepine, with an analgesic agent, such as an opioid, in a dosage form that results in high bioavailability and lack of delayed GI uptake due to lack of swallowing drug with saliva, therefore resulting in consistent onset and offset of drug action.

IV administration of opioids is typically used in acute and/or emergency medical situations to treat pain. It is rapid and effective, and pain relief is obtained within minutes. This route of administration of opioids requires a trained person to administer the medications, which may not be available in certain clinical or emergency situations. In such an acute and/or emergency medical situation in which IV access is difficult or impossible to obtain, anxiety coupled with the need for pain relief is typically treated by intramuscular injections of an opioid, such as morphine or meperidine. Oral tablets often take 30 minutes or longer to provide pain relief, which is too long for a pediatric patient in severe pain who is also suffering from anxiety.

Treatment of anxiety coupled with the need for pain relief is also necessary in many outpatient settings, such as prior to a potentially painful medical or dental procedure.

In the above settings, there is clearly a need for a rapid-acting dosage form that produces effective relief from anxiety and pain, and which may be used safely and conveniently.

A relatively undetectable dosage form that does not elicit a significant saliva response and therefore results in less swallowed drug and more drug taken up via the oral transmucosal route is a tremendous advantage in pediatric patients over current non-invasive drug delivery technology. One example is in pre-procedural sedation. One medication traditionally administered to pediatric subjects is oral midazolam, a cherry-flavored syrup (Versed syrup), currently FDA-approved for use in pediatric patients. Published studies demonstrate a delayed onset of action and an erratic bioavailability (30-50%) when midazolam is administered via this route. Delays in post-operative recovery have been reported due to slow GI uptake of the drug.

As mentioned above, transmucosal fentanyl and sufentanil have been shown to provide rapid-acting pre-procedural sedation in many studies. Intranasal sufentanil liquid has been studied in both adult and pediatric patients for pre-procedural sedation, with doses of 5-20 mcg or higher providing sedative effects (Vercauteren et al., Anaesthesia, 43:270-3, 1988; Karl et al., Anesthesiology, 76:209-15, 1992). There are some issues relating to onset of action and bioavailability, however, when the medication is inadvertently swallowed. For example, intranasal drops of sufentanil in adults result in 78% bioavailability due to swallowing of approximately one-third of the medication (Helmers et al., Canadian Journal of Anaesthesia, 36:494-7, 1989). As mentioned above, oral transmucosal fentanyl lozenge-on-a-stick (Oralet) was studied for use as a pre-procedural sedative and analgesic in pediatric patients undergoing central venous line removal (Wheeler et al., Pediatric Anesthesia, 12:594-599, 2002). Because of the large amount of drug swallowed, the onset of action was both delayed and erratic due to poor GI bioavailability of fentanyl and it was concluded that this fentanyl lozenge was not adequate for pre-procedural sedation in children. Therefore there is a need for transmucosal preparations of analgesic and sedative agents which do not result in inadvertent swallowing of the drug due to large saliva responses or nasal run-off.

A considerably higher and more consistent bioavailability has been demonstrated with oral transmucosal delivery of active drug using a NanoTab™. In a 12 subject clinical study in adults, delivery of sufentanil via a 5 microliter NanoTab™ dosage form in the sublingual space resulted in a bioavailability of greater than 90% with a coefficient of variation of less than 5% (Example 2, FIG. 2). Taste was rated as either undetectable or very mildly sweet by the majority of patients.

Medications, such as opioids, can be administered as single agents in the NanoTab™ for treatment of acute pain, or a combination of medications can be used in a single NanoTab™, such as a benzodiazepine and an opioid, for treatment of pre-procedural anxiety followed by post-procedural pain. Often in very short operative procedures, such as myringotomies (placement of ear tubes), only a mask induction of the young patient is performed for anesthesia and no IV is placed since this would significantly delay the operation. After awaking from the inhaled anesthetic, the child often has ear discomfort that is now difficult to treat due to lack of and IV catheter. A pre-operative opioid/benzodiazepine sublingual NanoTab™ would allow sedation for the mask induction and pain relief post-operatively. The high and consistent bioavailability of this dosage form would allow the anesthesiologist, surgeon and recovery room nurse to have less difficulty with pediatric patients in both the pre- and post-operative settings.

Sufentanil, alfentanil, remifentanil, lofentanil, carfentanil, trefentanil, and mirfentanil are potent fentanyl congeners that are rapidly metabolized and may be suitable for use in an oral transmucosal formulation in combination with an anxiolytic, such as triazolam.

In addition to fentanyl congeners, buprenorphine is an opioid with high transmucosal bioavailability and would be easily combined with a sedative, such as a benzodiazepine, for pre-procedural sedation.

The present invention provides a novel oral transmucosal dosage form that is relatively undetectable by the child and is effective to treat anxiety. In one approach, such a dosage form includes a fentanyl congener and a benzodiazepine which is effective to achieve synergistic sedative qualities, such that the standard dose of each drug can be lowered, thereby avoiding the complications due to a high dose of either drug.

The invention relies on small dosage forms comprising formulations for oral transmucosal drug delivery for the treatment of anxiety coupled with the need for analgesia.

In one exemplary application of the present invention, small dosage forms for oral transmucosal drug delivery are provided for inpatient or outpatient use to decrease the anxiety and pain associated with difficult intravenous access attempts. This is important for pediatric patients.

Compositions and methods of the invention for the treatment of anxiety disorders comprise a combination of a drug typically used to treat anxiety, e.g., a medication of the benzodiazepine class, together with a fentanyl congener. In one embodiment, the present invention provides for a combination formulation comprised of both an anxiolytic, such as a benzodiazepine, and a fentanyl congener, such as fentanyl or sufentanil, due to the synergy of these two classes of molecules in producing desired sedation without the untoward effects of a higher dosage of a single agent, such as fentanyl or sufentanil.

Oral transmucosal drug delivery for treatment of pre-procedural anxiety is described in U.S. Ser. No. 60/922,084, expressly incorporated by reference herein.

An exemplary dosage form for use in practicing the invention comprises from about 10 mcg to about 1000 mcg of triazolam or 0.5 mg to about 10 mg of midazolam together with about 1 mcg to about 1000 mcg of a fentanyl congener, e.g., from about 1 mcg to about 50 mcg of sufentanil or 10 mcg to about 500 mcg of fentanyl per dosage form for oral transmucosal delivery. As will be understood by those of skill in the art, the dose will be on the low end of the range for children and elderly patients and the high end of the range for adults dependent upon body mass.

In addition to opioids as analgesics, ketamine has analgesic properties and is a general dissociative anesthetic for human and veterinary use that is sold as the hydrochloride salt under the trade names: Ketanest, Ketaset, and Ketalar. Ketamine is classified as an NMDA receptor antagonist, and has a wide range of effects in humans, including analgesia, anesthesia, hallucinations, arterial hypertension, and bronchodilation. An exemplary dosage form for use in practicing the invention comprises from about 10 mcg to about 1000 mcg of ketamine, alone or in combination with an anxiolytic, such as a benzodiazepine.

### Nausea and Vomiting

Oral transmucosal delivery, e.g., by the sublingual route offers many theoretical advantages for drug administration when a patient is suffering form Nausea and/or vomiting and cannot swallow or keep an oral medication in the stomach long enough for the drug to be effective. Similar to patients whoa re “NPO” (restricted from oral input (food, liquids or medications), those who have severe nausea cannot ingest an oral medication. The oral transmucosal rout of drug administration, e.g., the sublingual route can be used for effective drug delivery in such patients, while lacking the invasive aspects of the intravenous route of administration.

Treatment of nausea may be accomplished using a drug selected from the group consisting of serotonin3 (5-HT3) receptor antagonists such as ondansetron, dolasetron and granisetron, dopamine receptor antagonists such as droperidol, prochlorperazine (Compazine®), metaclopramide and cannabinoid receptor agonists.

### Asthma

Asthma is characterized by coughing, wheezing and shortness of breath. This is typically treated with short-acting bronchodilators, such as short-acting beta-2 agonists, ipratropium (Atrovent) and/or oral or intravenous corticosteroids (prednisone, methylprednisolone, hydrocortisone and others).

However, the symptoms may persist, and become more marked leading to an acute asthma attack, characterized by an acute exacerbation of wheezing, unresponsiveness to usually effective therapy and necessitating care in an emergency room or hospital ward. An acute asthma attack is characterized by airways narrowing and inflammation, impairment of pulmonary function, alterations in alveolar ventilation and hypoxaemia.

Beta₂-adrenoreceptor agonists (beta₂ agonists) are typically used to treat acute asthma and act as short-acting bronchodilators. They are drugs that relax and open up the airways (bronchi) in the lungs, which become narrowed during an asthma attack. Examples of commonly used drugs are salbutamol (e.g. Ventolin, Volmax) and terbutaline (Bricanyl).

Asthma medications are generally available as inhaled treatments that deliver medication directly to the airways, typically using a nebulizer or metered dose inhaler (MDI).

A nebulizer includes a source for compressed air such that the air flow to the nebulizer changes the medication solution to a mist, which is inhaled for approximately 5-10 minutes. A face mask may be used with a nebulizer for very young children.

A metered-dose inhaler (MDI) is a handheld device that delivers a measured dose of medication directly to your lungs. The medication is usually in an aerosol form.

Administration of asthma medications by inhalation is not effective if the airways are constricted and the medication cannot quickly or effectively be delivered to the lungs.

The invention includes dosage forms for treatment of asthma delivered by the oral transmucosal route. The dosage forms comprise terbutaline or other beta₂-adrenergic receptor agonist which is delivered via a small transmucosal dosage form such that minimal swallowing of drug occurs and rapid consistent transmucosal uptake of the drug results in rapid bronchodilation.

Examples of short-acting beta₂agonists include but are limited to, terbutaline (10 mcg-2 mg), salbutamol (10 mcg-2 mg), albuterol (200 mcg-10 mg), fenoterol (10 mcg-2 mg) and orciprenaline (200 mcg-10 mg).

V. Dosage Forms

The present invention provides the advantage of a dosage form that is acceptable to pediatric subjects, where the medication being administered has a relatively fast onset. The medication is provided in a form where unpleasant taste can be masked in order to avoid crying and/or distress on the part of the pediatric subject.

The dosage forms of the invention are provided for transmucosal drug delivery and can have bioadhesive characteristics. The dosage forms typically adhere to a mucosal membrane such as a sublingual or buccal membrane and may be provided in the form of a lozenge, pill, tablet, capsule, membrane, strip, liquid, patch, film, suppository, gel or spray.

The present invention provides a novel dosage form, the NanoTab™, which is specifically designed so as to not be detectable by the patient once placed in the mouth, such as under the tongue in the sublingual space. Small drug dosage forms or NanoTabs™ for use in practicing the present invention are described in U.S. Ser. No. 11/650,174, expressly incorporated by reference herein. The NanoTab™ consists of a very small size dosage form that lies flat on the mucosal surface and may also contain a bioadhesive quality to avoid inadvertent dislodging by movements of the tongue.

Exemplary NanoTabs™ for administration to pediatric subjects, i.e., infants and young children have a volume of less than 30 ul, e.g., a mass of less than 5 ul, 6 ul, 7 ul, 8 ul, 9 ul, 10 ul, 11 ul, 12 ul, 13 ul , 14 ul , 15 ul, 16 ul, 17 ul, 18 ul, 19 ul, 20 ul, 21 ul, 22 ul, 23 ul, 24 ul, 25 ul, 26 ul, 27 ul, 28 ul, 29 ul or 30 ul.

In another embodiment, exemplary NanoTabs™ for administration to infants and young children have a mass of less than 30 mg, e.g., a mass of less than 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg or 30 mg.

A further advantage of the NanoTab™ is that the small size of the dosage form produces a minimal saliva response. This is important since commercially available transmucosal medications, such as Actiq, Oralet, and Fentora, only benefit from 25-50% of the drug being taken up by the mucosa since the large saliva response that occurs with these large dosage forms (>100 microliters in volume) results in swallowing of the active drug. This in turn results in an erratic and slow onset of the desired response, in this case pain relief, due to the GI uptake of the majority of the drug. This also results in a low and erratic bioavailability of the drug (33-65%). By utilizing a very small volume dosage form (for example, less than 30 microliters or less than 30 mg), more of the drug is available for transmucosal delivery and less is solubilized in saliva and swallowed.

The oral dosage form is typically a “sublingual dosage form”, but in some cases other oral transmucosal routes may be employed. The preferred site for drug delivery is the sublingual area, although in certain embodiments it may be advantageous for the dosage form to be placed inside the cheek, or to adhere to the roof of the mouth or the gum.

Due to the relative lack of taste buds in the sublingual space, the taste of the medication is also minimal, further reducing saliva production and also making it less likely to allow detection of the dosage form by pediatric patients.

The oral transmucosal dosage forms of invention are designed to fit comfortably under the tongue such that the drug form disintegrates over the appropriate time period. The dosage forms are designed to provide for rapid onset of efficacy, while avoiding the immediate peak plasma levels followed by significant drop off seen in prior art formulations such as described in U.S. Pat. No. 6,759,059, wherein fentanyl was administered via tablets containing 400 μg of fentanyl which resulted in a peak plasma level of 2.5 ng/ml at 5 minutes post-administration, followed by an immediate drop in plasma level.

Additional advantages of the present invention are the ability of the drug formulation to provide higher levels of drug absorption via the oral transmucosal route. The present invention provides an oral transmucosal dosage form, exemplified herein by the NanoTab™ that provides for high bioavailability, low variability in Tₘₐₓ, low variability in Cₘₐₓ and low variability in AUC. The NanoTab™ dosage forms of the invention also provide for controlled dissolution, solubility and stability, resulting in controlled release of the drug over time resulting in prolonged plasma levels within the therapeutic window, making the current invention a significant improvement for the treatment of pediatric subjects.

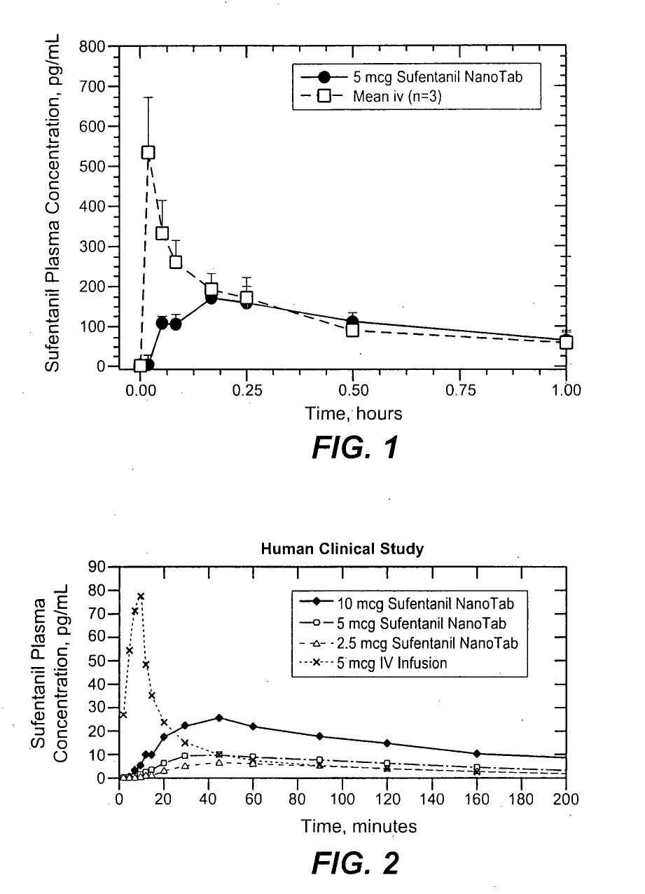

As detailed in Example 1, a Beagle dog study was carried out to compare a sublingual 5 mcg sufentanil dosage form (NanoTab™) to IV sufentanil. This study was also performed to demonstrate the ability of an uncooperative mammal to achieve superior drug administration via the undetectable sublingual dosage form of a 5 microliter NanoTab™. A total of three Beagle dogs were studied and the results are graphed in FIG. 1. The bioavailability of the sublingual sufentanil NanoTabs™ was 75% compared to IV. The high bioavailability data in dogs confirms the superior attributes of the NanoTab™ over larger dosage forms. Furthermore, the coefficient of variation for the AUC was low, 14%, compared to the variation of other commercial transmucosal dosage forms, as further described below.

As detailed in Example 2, a human study was carried out to compare sufentanil NanoTabs™ containing either 2.5 μg, 5 μg or 10 μg of sufentanil base corresponding to 3.7 μg, 7.5 μg or 15 μg of sufentanil citrate, respectively, to IV sufentanil. The NanoTab™ dosage forms in this study eroded over a period of 10-30 minutes in all subjects. After placement of each sufentanil sublingual NanoTab™ in the sublingual space of the 12 healthy volunteers, a remarkably consistent pharmacokinetic profile was obtained (FIG. 2). The bioavailability compared to IV administration for single administration of all three dosages averaged 91%, which is far superior to that measured for commercially available fentanyl transmucosal preparations, Actiq and Fentora (47% and 65%, respectively—Fentora package insert). Although this high bioavailability could be due to a number of factors, it is likely that the lack of saliva produced by the small NanoTab™ size significantly limits the swallowing of the drug and avoids the low bioavailability typical of drug absorption via the GI route. Currently available fentanyl products, Fentora and Actiq, have package inserts which claim at least 50% and 75% of the drug dose, respectively, is swallowed via the saliva, and therefore both exhibit lower bioavailability than the NanoTabs™ of the invention. The NanoTabs™ used in this clinical trial had a volume of approximately 5 microliters (mass of 5.5 mg), a small fraction of the size of Actiq or Fentora lozenges. The dog and human studies described in Examples 1 and 2, demonstrate that although sufentanil has very poor GI bioavailability (12%), the observed high bioavailability following administration of sufentanil NanoTabs™, wherein drug is administered by the oral transmucosal route, supports the conclusion that greater than 75% of the drug is absorbed transmucosally,. Therefore, less than 25% of the drug is swallowed, illustrating the superiority of the compositions and methods of the present invention, as compared to Fentora or Actiq.

Importantly, this high bioavailability is also linked to high consistency of total drug delivered to the patient. For example, the total plasma drug area under the curve (AUC 0-infinity) for sufentanil NanoTabs™ 10 mcg was 0.0705±0.0194 hr*ng/ml (mean±standard deviation (SD)).This SD is only 27.5% of the total AUC. Coefficient of variation (CV) is a term to describe the percent SD of the mean. The coefficient of variation for Fentora AUC is 45% and for Actiq AUC is 41% (Fentora package insert). Therefore the total dose delivered to the patient/subject is not only more bioavailable for the sufentanil NanoTabs™ but it is more consistently the same from patient to patient.

The sufentanil sublingual NanoTabs™ are also superior in terms of consistent drug plasma levels early after administration. The Cₘₐₓ obtained with the 10 mcg sufentanil NanoTab™ was 27.5±7.7 pg/ml. The coefficient of variation of the Cₘₐₓ therefore is only 28%. The Cₘₐₓ for Fentora and Actiq suffer from variability of GI uptake of drug. Fentora reports a Cₘₐₓ of 1.02±0.42 ng/ml, therefore the coefficient of variation of the Cₘₐₓ is 41%. The range of coefficients of variation for the various doses of Fentora is from 41% to 56% (package insert). Actiq coefficient of variation of Cₘₐₓ is reported as 33% (Fentora package insert).

### Formulations

The dosage forms for use in practicing the present invention is a substantially homogeneous composition which comprises active ingredients and one or more bioadhesives that provide for adhesion to the oral mucosa, e.g., a sublingual or buccal membrane of a pediatric patient, one or more binders that provide binding of the excipients in a single dosage form, one or more hydrogel-forming excipients, one or more bulking agents, one or more lubricants, as well as other excipients and factors that modify and control the drug's dissolution time and kinetics or protect the active from degradation.

Formulations for making small volume bioadhesive drug dosage forms for oral transmucosal administration to a pediatric subject comprise a predetermined amount of a pharmaceutically active drug and a bioadhesive material and are characterized by a bioadhesion force of greater than 100 dynes/cm², e.g. 500 dynes/cm².

Formulations for use in practicing the present invention and methods for evaluating the bioadhesion force are described in U.S. Ser. No. 11/650,227, expressly incorporated by reference herein.

In one aspect, a drug dosage form applicable to the present invention comprises from 25 μg to 99.9 mg, from about 1 μg to 50 mg, or from about 1 μg to 10 mg of the drug. The dosage forms of the invention have a high content uniformity.

The invention further provides drug dosage forms which have an erosion time of from about 1 second up to a time selected from 1 minute, 2 minutes, 3 minutes, 4 minutes, 5 minutes, 10 minutes, 15 minutes, 30 minutes or longer.

In certain embodiments of the invention, the drug dosage form is adapted to deliver 30% or more of the total amount of drug contained in a single drug dosage form to an individual via the oral mucosa. The percentage of the total amount of drug contained in a single drug dosage delivered transmucosally is greater than 30-40%, 40-50%, 60-70%, 70-80%, 80-90% and preferably greater than 95%. In exemplary embodiments, at least 35%, 40%, 45%, 50%, 55%, 60%, 65%, 70%, 75%, 80%, 85%, 90%, 95%, 98% or 99%, of the total amount of drug contained in a single drug dosage form is delivered via the oral mucosa.

The drug dosage form is also adapted to deliver no more than 60% of the total amount of drug contained in a single drug dosage form to an individual via the GI tract. Typically, the percentage delivered via the GI tract maybe lower, such that not more than 50%, 40%, 30%, 20%, 10%, 5% or 1% of the total amount of drug contained in the drug dosage form is delivered to the individual via the GI tract.

The delivery of a greater percentage (and amount) of drug via the oral mucosa and the corresponding lack of delivery via the GI tract provides a significant improvement over standard oral drug delivery where the drug is swallowed.

### Single and Multiple Dose Applicators

In practicing the invention, small volume dosage forms are administered to the oral mucosa of a pediatric subject with or without a device, for example using a single or multiple dose applicator.

The invention provides disposable applicators for delivering dosage forms to the oral mucosa such that manual application to a pre-determined location for drug delivery (e.g. the mouth, sublingual space, etc.) is effected.

To further improve pediatric patient acceptance of this NanoTab™ dosage form, colorful and decorative single-dose administration (SDA) devices could be used to administer the NanoTab™ to children. Because the NanoTab™ is very small and thin, handling this dosage form can be difficult without the aid of an applicator. In the case of pediatric patients, this applicator could be designed to be appealing to young patients, thereby calmlng their anxiety about administration of the drug form.

A small dosage form or NanoTab™ may be delivered to the oral mucosa, for example, using a single dose applicator. The applicator may be designed to be “child friendly” by having a design or decoration that is attractive to a child, such as a flower or animal or character seen on television or in a children's book.

A small dosage form or NanoTab™ is provided in a child-resistant dispenser or packaging and delivered to the sublingual space with supervision or administered with assistance with or without a device.

In one embodiment, a single dose applicator is used for a variety of drug dosage forms, including a solid tablet, a liquid capsule, a gel capsule, a liquid, a gel, a powder, a film, a strip, a ribbon, a spray, a mist, a patch, or any other suitable drug dosage form.

The single dose applicator (SDA) may contain the dosage form within, may have the drug dosage form attached or affixed to it, may afford a seal against moisture, humidity, and light, and may be manually manipulated by a patient, healthcare provider, or other user to place said dosage form in the proper location for drug delivery.

For delivery to a pediatric subject, a healthcare provider or parent will place the dosage form in the subject's mouth or supervise placement by the subject.

In practicing the invention, a single- or multiple-dose applicator or drug dispensing device may be used to deliver tablets or other dosage forms into the hand, the mouth, under the tongue, or to other locations appropriate for specific drug delivery needs.

In one embodiment, a single- or multiple-dose applicator or drug dispensing device is used to deliver a dosage form to the oral mucosa, e.g., the sublingual space.

The dosage forms inside the dispensing device should remain dry prior to dispensing, at which point a single dosage form is dispensed from the device into the mouth, e.g., the sublingual space, wherein a patient's saliva will wet the dosage form and allow for disintegration/erosion and drug dissolution.

The SDA may be provided as a pair of forceps, a syringe, a stick or rod, a straw, a dropper, a sprayer or atomizer, or any other form suitable for the application of a single drug dosage form. After use, the SDA may be disposed of, so as to eliminate the risk of contaminating the dispenser with saliva, or other contaminants.

In one aspect of the invention, a small volume dosage form according to the present invention is placed in the sublingual space, preferably under the tongue on either side of the frenulum linguae, such that it adheres upon contact. While not wishing to be bound by theory a dosage form of the invention is exposed to the moisture in the sublingual space, resulting in the formation of a hydrogel network.

For sublingual administration, a small volume dosage form may be administered sublingually by placement under the tongue, adjacent to the frenulum using forceps. Alternatively, a small volume dosage form may be administered sublingually by placement under the tongue, adjacent to the frenulum using a syringe, a stick or rod, a straw, a dropper, or any other form suitable for the application of a single drug dosage form, including but not limited to a SDA, as further described herein.

In another embodiment, a dispenser of the invention may contain a plurality of SDA's, in a cartridge or individually packaged, and may dispense a single SDA containing a single drug dosage form for use by the patient, healthcare provider, or user. The dispenser may dispense single SDA's in the same way and with the same features as would be advantageous for the dispensing of single drug dosage forms described in the invention.

The dosage forms may be provided in a package that consists of molded plastic or laminate that has indentations (“blisters”) into which a dosage form, is placed, referred to herein as a “blister pack”. A cover, typically a laminated material or foil, is used to seal to the molded part. A blister pack may or may not have pre-formed or molded parts.

In one embodiment, the blister pack has two flexible layers that are sealed with the dosage form in between and the primary unit dose blister pack also serves as an applicator for delivering a single dosage form to the sublingual space, once the child-resistant foil is peeled back.

In yet another embodiment of the invention, a long tape or array of dosage forms sealed between a flexible blister layer and a foil or otherwise breakable layer is provided. A pusher is positioned above a dosage form, and upon actuation pushes against the blister, forcing the dosage form through the foil or breakable layer, dispensing the dosage form.

Such blister packs may be provided in a child resistant multiple dosage dispenser.

The general use of blister packs for dispensing medications is known. For example, U.S. Pat. No. 5,348,158 (Honan et al.) discloses a reusable dispensing package for the successive dispensing of tablets, pills and capsules in a predetermined sequence. A blister pack containing the medication is placed in a hinged container which rotates such that a tablet, pill or capsule contained in the blister pack is released in a predetermined sequence.

U.S. Pat. No. 5,489,025 (Romick) discloses a dispenser for dispensing unit doses of medication from a blister pack. The dispenser includes a top plate and a bottom plate which holds the blister pack, where the top plate has a face area and a back area with at least one aperture for receiving a blister of a blister pack and the bottom plate has at least one dispensing aperture in register with the blister of the blister pack.

Child resistant multiple dosage blister pack dispensers wherein a bottom panel has a plurality of orifices each housing individual dosages above an orifice, such that one can push individual dosages from the blister pack through an orifice have been described for example in U.S. Pat. No. 6,726,053 (Harrold).

Single patient dose medicament dispensers have been described wherein a single dose disposable dispenser comprising a tray with wells containing the substance or substances to be administered are provided together with an applicator where a portion of the applicator extends beyond the open end of the applicator well. The applicator well is uncovered or squeezed to extrude the substance or substances to be administered. See, e.g., U.S. Pat. Nos. 5,660,273; 6,959,808; 6,116,414; 6,328,159.

In another approach electronic foil circuitry mounted on the lidstock of a blister package is used to detect dosing from a blister pack (U.S. Patent Publication No. 20050122219).

Although these patent publications disclose blister packs for dispensing medications, such dispensers would not be effective to deliver a small volume dosage form to the oral mucosa.

This permits the handling of only a single drug dosage form at a time and prevents the other individually sealed drug dosage forms from becoming exposed to saliva, humidity and the like.

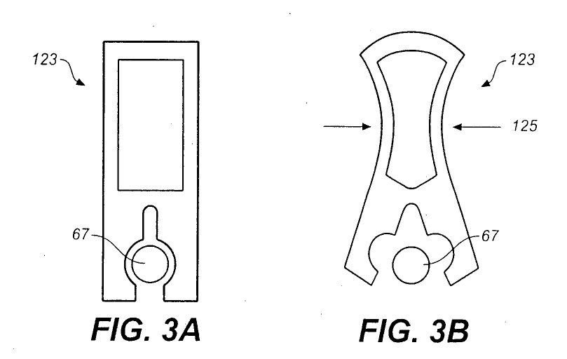

FIGS. 3A and 3B show one embodiment of a single dose applicator 123 a dispensing device for delivering drug dosage forms. The dispensing device shown in FIG. 14A depicts the single dose applicator 123 that is ready to dispense a drug dosage form 67. In one aspect of this embodiment, a user pinches the single dose applicator 125 which opens the applicator and a drug dosage form 67 is dispensed as shown in FIG. 14B.

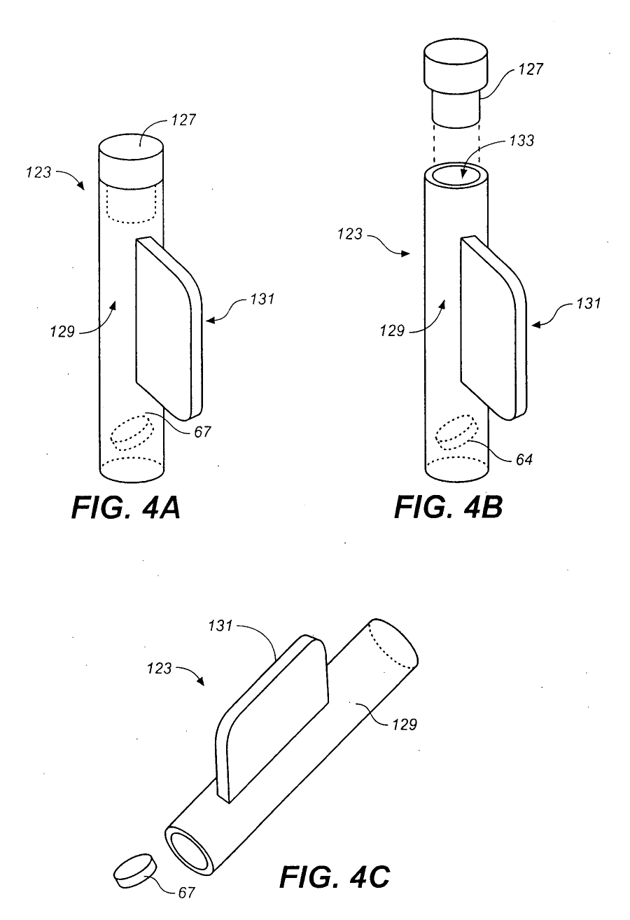

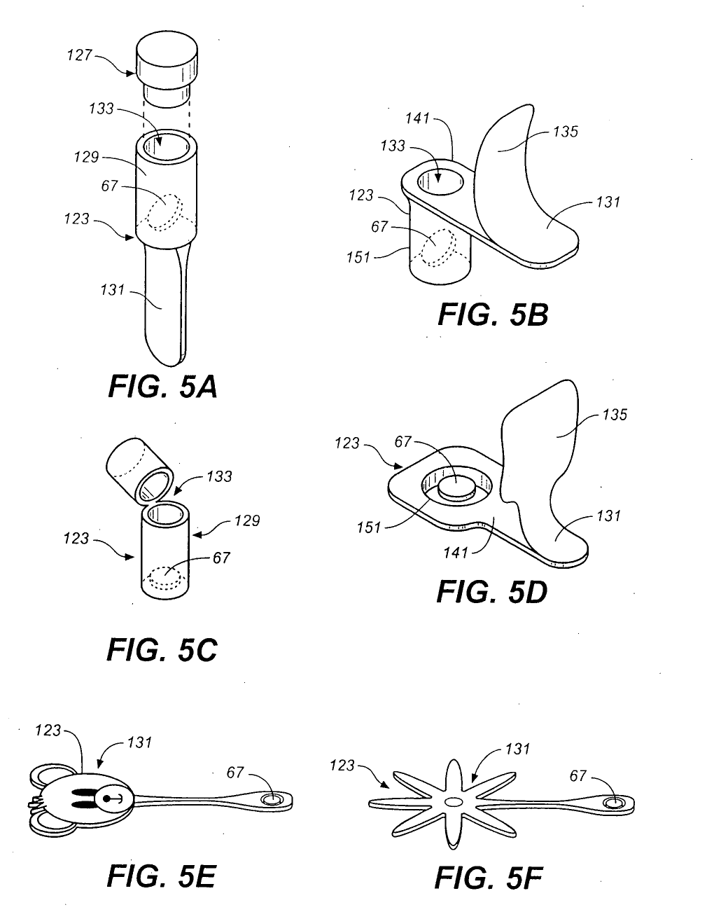

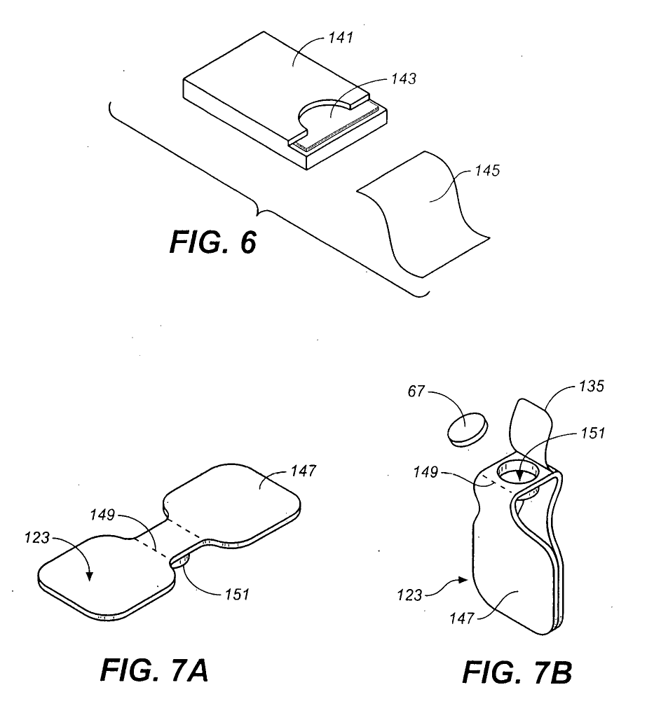

FIGS. 4A-C, FIGS. 5A-F, FIG. 6 and FIGS. 7A and B are schematic depictions of exemplary embodiments of a SDA of the invention.

FIGS. 4A-C show an embodiment of a single dose applicator 123 that is comprised of a applicator shaped as a tube 129, a stopper seal 127, a handle 131 (e.g., an ergonomic handle), and a single dosage form 67. FIG. 4A shows the single dose applicator 123 in its sealed configuration, prior to use. FIG. 4B shows the single dose applicator 123 with its stopper seal 127 removed, forming an opening 133, and ready for use. FIG. 4C shows the single dose applicator 123 tilted so as to dispense the dosage form 67 adjacent the oral mucosa, e.g., in the sublingual space.

FIGS. 5A-F show several alternate embodiments of the single dose applicator 123. In all of these figures the applicator seal 127 is broken and the applicator is tilted so as to drop the drug dosage form 67 adjacent an oral mucosal membrane in the mouth of a subject, e.g., under the tongue for sublingual dosage form placement. FIG. 5A shows a tube like applicator 129 with a handle 131 located axially under the tube 129. FIG. 5B shows an applicator formed as a thermoform or blister package 151 with a foil seal 135 that is peeled so as to open the applicator package 141 prior to placing the dosage form 67. FIG. 5C shows an applicator that is a tube 129 which is broken to break the seal prior to dosage form 67 placement. FIG. 5D shows a blister pack tube 151 type dosage form package 141 with a handle 131 such that after the seal 135 is peeled back the blister pack 151 can be held and tilted to place the drug dosage form 67, adjacent an oral mucosal membrane. FIGS. 5E and 5F show blister pack 151 type packaging with a handle 131 shaped like a flower or an animal, respectively, to be used for single dose applicator 123 designed for pediatric use. Other single dose applicator shapes could include cartoon characters, animals, super-heroes or other appropriate shapes for pediatric applications.

The handle may be decorative and/or “child friendly”, as exemplified in FIG. 5E and 5F, in order to make the SDA more appealing to the pediatric subject.

FIG. 6 shows dissolving film dosage forms 145 and a dosage form package 141 with a plurality of dissolving film dosage forms 143 within it. The dissolving film dosage form 143 is removed from the package 141 and placed adjacent an oral mucosal membrane, e.g., in the sublingual space where it dissolves and delivers the drug transmucosally.

FIGS. 7A-B provides illustrations of two stages of use of one embodiment of a single dose applicator 123. FIG. 7A shows the applicator 123 in its configuration prior to use, with two applicator tabs 147, two perforations 149, and a blister pack 151 containing a dosage form 67. In order to administer the dosage form 67, the two applicator tabs 147 are bent downward at the perforations 149, forming a handle 131, and the seal 135 is peeled back to reveal the blister pack 151 and allow the dosage form 67 to be dropped adjacent an oral mucosal membrane, e.g., in the sublingual space.

V. Devices for Administration of Oral Dosage Forms to a Pediatric Subject

The present invention further provides dispensing devices for delivery of a drug dosage form to the oral mucosa of a pediatric subject, wherein the device comprises a means to dispense multiple doses, a single dose at a time.

In one embodiment, a drug dispensing device may contain a plurality of SDA's, in a cartridge or individually packaged, and may dispense a single SDA containing a single drug dosage form for administration to a pediatric patient. The drug dispensing device is child-resistant and may dispense single SDA's in the same way and with the same features as would be advantageous for the dispensing of single drug dosage forms described in the invention.

All publications mentioned herein are incorporated herein by reference in their entirety for the purpose of describing and disclosing the compositions and methodologies which are described in the publications which might be used in connection with the presently described invention. The publications discussed herein are provided solely for their disclosure prior to the filing date of the present application. Nothing herein is to be construed as an admission that the invention is not entitled to antedate such a disclosure by virtue of prior invention.

### EXAMPLES

### Example 1

### In Vivo Evaluation of Sublingual Sufentanil NanoTabs™ in a Dog Model

The following Example relies on a Beagle dog model and the formulations for the NanoTab™ dosage forms with a total mass of 5.5 mg. The in vivo pharmacokinetics (PK) of sufentanil following sublingual administration of an exemplary 5 mcg NanoTab™ formulations were evaluated in a healthy Beagle dog model. The formulations are described in U.S. Ser. No. 11/650,174, expressly incorporated by reference herein. Briefly, single 5 mcg NanoTabs™ were administered sublingually in fully awake healthy dogs by direct placement in the sublingual space. This model approximates the conditions in the pediatric setting where the child, similar to a conscious animal, may not cooperate with administration of a dosage form that was detectable. A total of three dogs were evaluated. Following administration, the position of the NanoTab™ in the sublingual space was observed visually at 5-15 minute intervals following administration. The sublingual sufentanil PK was compared with that of IV administered sufentanil at the same dose level.

All dogs were catheterized via the cephalic vein for blood collections up to 2 hours post-dosing. Through the 2-hour post-dose blood collection, all dogs were fitted with an Elizabethan collar to prevent removal of the catheter. The catheter was removed following the 2-hour blood collection. The 4-, 8-, and 24-hour post-dose blood collection were collected from the cephalic or other suitable vein. Approximately 2 ml of blood were collected into pre-chilled tubes containing potassium EDTA at the following timepoints: prior to dosing and approximately 1, 3, 5, 10, 15, 30 min 1, 2, 4, 8 and 24 hours post-dose. The samples were analyzed with the appropriately validated LC/MS/MS method for the determination of sufentanil citrate in dog plasma. The sufentanil plasma concentrations and the pharmacokinetic results are shown in FIG. 1.

### Example 2

### In Vivo Evaluation of Sublingual Sufentanil NanoTabs™ in a Human Study

A human clinical study was performed using healthy volunteers to demonstrate the more consistent Tₘₐₓ, Cₘₐₓ and bioavailability when small dosage forms that do not stimulate a saliva response are utilized sublingually. The study was performed with 12 subjects (6 men and 6 women) using sufentanil exemplary NanoTab™ formulations, manufactured to have a volume of 5 μL, a mass of approximately 5.5 mg, and determined to have a uniform size for all dosage strengths with dimensions of approximately 3 mm in diameter and 0.8 mm in thickness. The formulations are described in U.S. Ser. No. 11/650,174, expressly incorporated by reference herein. Sufentanil NanoTabs™ contained either 2.5 μg, 5 μg or 10 μg of sufentanil base corresponding to 3.7 μg, 7.5 μg or 15 μg of sufentanil citrate, respectively. All excipients were inactive and have GRAS (“generally recognized as safe”) status. The sufentanil NanoTabs™ were tested for sublingual use. Study staff administered individual NanoTabs™ to a subject by placing them directly at the base of the frenulum using blunt-tipped forceps.

The NanoTabs™ for this study eroded over a period of 10-30 minutes in all subjects. After placement of each sufentanil sublingual NanoTabs™ in the sublingual space of the 12 healthy volunteers, a remarkably consistent pharmacokinetic profile was obtained for the three dosages (FIG. 2).

### Example 3

A pediatric patient is scheduled for a myringotomy procedure. The 4 year-old child is anxious and does not allow the anesthesiologist to easily perform a mask induction and no IV access is available. A 5 microliter volume NanoTab™ containing a combination of sufentanil 15 mcg and triazolam 200 mcg is administered under the child's tongue using a plastic applicator. Since the child cannot taste or detect the presence of the NanoTab he does not try to spit out or swallow the dosage form. Within 10 minutes the child becomes sedated enough to allow an easy mask induction. After the short 20 minute procedure, the child does not complain of pain since the sufentanil is effective in treating the mild post-operative pain as well.

### Example 4

A young 4 year-old child is playing soccer and develops severe respiratory distress due to exercise-induced asthma. She is not inhaling enough air to effectively use a metered-dose inhaler and she also is too anxious to cooperate with her parents as they try to encourage her use of the MDI. Instead they place a sublingual NanoTab™ which contains 200 mcg of the beta adrenergic agonist terbutaline under the child's tongue. It dissolves within 1 minute and the transmucosal uptake of the drug avoids the delay of GI uptake and therefore the drug begins to work within 5 minutes to bronchodilate her lungs.

### Example 5

A young child is in the emergency room with a broken arm. She is 3 years old and is in too much pain for the physician to perform a physical exam. To avoid the additional discomfort of placing an IV to administer analgesics, a sublingual sufentanil NanoTab™ containing 10 mcg of sufentanil is administered under the patient's tongue. She can't detect the small dosage form and therefore does not spit our or swallow the dosage form. Within 10 minutes the patient stops crying and the physician can perform an exam and take x-rays.

### Example 6

A 6 year-old boy is recovering from a major orthopedic surgery and his IV infiltrates. He has no need for IV access other than for analgesic medications. His doctor switches his analgesic regimen from IV patient-controlled morphine to patient-controlled analgesia using sufentanil (5 mcg) NanoTabs™ sublingually. The NanoTabs™ are dispensed from a device which has a 20 minute lock-out programmed so as to avoid overdosing. Since the n use this sublingual route of administration at home, no transition of analgesics is upon discharge from the hospital.

1-77. (canceled)

78. A small volume bioadhesive drug dosage form for oral transmucosal administration to a pediatric subject, comprising:
(a) a pharmaceutically active amount of a drug; and
(b) a bioadhesive material, wherein said bioadhesive material provides for adherence to the oral mucosa of said pediatric subject and said dosage form has a mass of less than 15 mg or a volume of less than 15 μl.

79. The small volume dosage form according to claim 78, wherein said dosage form has a mass of less than 8 mg or a volume of less than 8 μl.

80. The small volume drug dosage form according to claim 78, wherein said oral transmucosal administration is sublingual administration.

81. The small volume drug dosage form according to claim 78, wherein said oral transmucosal administration is buccal administration.

82. The dosage form according to claim 78, wherein said dosage form comprises from about 1 μg to 10 mg of said pharmaceutically active drug.

83. The dosage form according to claim 82, wherein said pharmaceutically active drug is sufentanil, alfentanil, lofentanil, carfentanil, remifentanil, trefentanil, mirfentanil, triazolam, alprazolam, midazolam, temazepam, estazolam, flurazepam, nitrazepam, bromazepam, halazepam, clonazepam ondansetron, dolasetron, granisetron, droperidol, prochlorperazine, metaclopramide, a cannabinoid receptor agonist, salbutamol, terbutaline, albuterol, fenoterol, orciprenaline, zolpidem tartrate, zaleplon, eszopiclone, or a combination thereof.

84. The dosage form according to claim 83, wherein said pharmaceutically active drug is triazolam, alprazolam, midazolam, temazepam, estazolam, flurazepam, nitrazepam, bromazepam, halazepam or clonazepam.

85. A method for anxiety in a pediatric subject, comprising:
administering a drug dosage form according to claim 7 to said subject, wherein the drug dosage form is not detected by the subject and following said administration, relief from anxiety is evident.

86. The method according to claim 85, wherein said drug dosage form is administered with assistance.

87. The method according to claim 85, wherein said drug dosage form is administered using a device.

88. The method according to claim 85, wherein said drug dosage form is administered using a single dose applicator.

89. The method according to claim 85, wherein said oral transmucosal administration is sublingual administration.

90. The dosage form according to claim 83, wherein said pharmaceutically active drug is ondansetron, dolasetron, granisetron, droperidol, prochlorperazine, metaclopramide, a cannabinoid receptor agonist or a combination thereof.

91. A method for treating nausea or vomiting in a pediatric subject, comprising:
administering a drug dosage form according to claim 90 to said subject, wherein the drug dosage form is not detected by the subject and following said administration, relief from nausea or vomiting is evident.

92. The method according to claim 91, wherein said drug dosage form is administered with assistance.

93. The method according to claim 91, wherein said drug dosage form is administered using a device.

94. The method according to claim 91, wherein said drug dosage form is administered using a single-dose applicator.

95. The dosage form according to claim 83, wherein said pharmaceutically active drug is salbutamol, terbutaline, albuterol, fenoterol, orciprenaline, or a combination thereof.

96. A method for treating asthma in a pediatric subject, comprising:
administering a drug dosage form according to claim 95 to said subject, wherein the drug dosage form is not detected by the subject and following said administration, relief from asthma is evident.

97. The method according to claim 96, wherein said drug dosage form is administered with assistance.

98. The method according to claim 96, wherein said drug dosage form is administered using a device.

99. The method according to claim 96, wherein said drug dosage form is administered using a single-dose applicator.

100. The method according to claim 96, wherein said oral transmucosal administration is sublingual administration.

101. The dosage form according to claim 78, wherein the amount of said pharmaceutically active drug delivered transmucosally is a percentage selected from the group consisting of at least 35%, at least 40%, at least 45%, at least 50%, at least 55, at least 60%, at least 65%, at least 70%, at least 75%, at least 80%, at least 85%, at least 90%, at least 95%, at least 98% and at least 99% of the total amount of pharmaceutically active drug in said dosage form.

102. The dosage form according to claim 101, wherein the amount of pharmaceutically active drug delivered transmucosally is at least 35% of the total amount of pharmaceutically active drug in said dosage form.
